(19) United States
(12) Patent Application Publication
(10) Pub. No.: US 2008/0038305 A1
(43) Pub. Date: Feb. 14, 2008

(54) Ceramic Bulking Agent

(76) Inventors: Jacobus Adriaan Albertus Kotze, Pretoria (ZA); Hermanus Lambertus Kotze, Kwazulu Natal (ZA); Stuart Kenneth Jackson Smyth, Essex Junction, VT (US)

Correspondence Address: NIXON & VANDERHYE, PC, 901 NORTH GLEBE ROAD, 11TH FLOOR, ARLINGTON, VA, 22203, US

(21) Appl. No.: 11/578,669

(22) PCT Filed: Apr. 25, 2005
(86) PCT No.: PCT/IB05/51335

§ 371 Date: Jul. 16, 2007

(30) Foreign Application Priority Data
Apr. 23, 2004 (ZA) 2004/3126

(51) Int. Cl. A61K 9/14; A61K 33/08; A61K 33/42; A61P 13/00; A61K 9/00

(52) U.S. Cl. 424/422; 424/400; 424/489; 424/600; 424/602; 424/691

(57) ABSTRACT

This invention relates to a tissue bulking agent comprising a particulate micro-porous bioinert ceramic material, in the form of spheres, having interconnected pores. Pores on the surface of each sphere are connected to pores inside such sphere via blow-holes and the internal pores are in turn interconnected, so that in addition to a high porosity, the spheres have a high permeability to gas and liquid. The bulking agent is disposed inside a syringe and is activated by impregnating the particles with traces of a bioactive resorbable bioceramic or by the in situ impregnation with serum from the blood of a patient. The particles are mobilised for injection by mixing the particles with a bio-suitable carrier gel or a viscous, lubricating carrier liquid. Thereafter, the activated and mobilised bulking agent is injected into the tissue in the area to be treated or bulked, such as the pelvic floor or urinary sphincter.

### INTRODUCTION

This invention relates to a tissue bulking agent and a device for bulking a portion of tissue in vivo. This invention further relates to a method and means for treating urinary incontinence related disorders. More particularly, but not exclusively, this invention relates to an injectable ceramic composite for use as a tissue bulking agent in the human body.

### BACKGROUND TO THE INVENTION

The involuntary loss of urine, known as urinary incontinence, is a significant problem affecting over 45 million men and women around the world. The failure to store urine properly is typically a result of a malfunctioning bladder or urethra. Some of the most severe cases of both male and female incontinence are those resulting from a poor or malfunctioning bladder mechanism. Incontinence may be caused by a lack of anatomic support of the urethra allowing it to move out of the abdominal cavity or by intrinsic deficiency of the urethral closure mechanism. A normal urethra will not leak at any physiologic abdominal pressure, but individuals who suffer from incontinence, may experience leakage during, for example, coughing or straining. Therefore, proper treatment of urinary incontinence is directed towards methods of achieved urethral resistance to changes in abdominal pressure.

Before 1995, when injectable collagen was introduced, there were virtually no options for the millions of women with stress incontinence other than invasive surgery. Collagen is injected into tissue, such as the urinary sphincter, to increase resistance to the flow of urine. This method suffers from some serious disadvantages. Collagen is water-soluble and is therefore absorbed by the body, so that repeated treatments are necessary. This makes the cost of collagen treatments virtually prohibitive. In addition, some patients develop allergic reactions that include rashes, swelling, erythema and anaphylactic shock. Collagen injections have also been used to augment other tissue, such as lip tissue in order to provide the lips with a fuller appearance. However, this procedure has similar limitations to the collagen-treatment of incontinence, namely high cost, absorption of collagen by the body and allergic reactions to the treatment.

Another known material used to treat incontinence is carbon-coated beads. Recent double blind randomised studies comparing carbon-coated beads to collagen, however, reported only equal effectiveness or a similar poor level of performance. There was also no difference in the number of treatments between the two groups.

Within North America, materials such as collagen, carbon-coated beads and Teflon were intended to treat the millions of patients who specifically had intrinsic sphincter deficiency, where total closure of the sphincter is not achieved. Eighty two percent of the female stress incontinent patients' conditions were attributed to urethral hyper mobility, where the urethra moves or tugs on the sphincter when abdominal pressure is applied, allowing urine to leak. Until recently, bulking the sphincter has not been indicated for the treatment of hyper mobility, and leading academics report that mild to moderate stress incontinence is not entirely due to one or the other condition. It has been reported that physicians rarely see hyper mobility without intrinsic sphincter deficiency and that in fact they believe they are not separate entities, which suggests that hyper mobility is not the cause for incontinence. The outcome of their work emphasises the likelihood of achieving coaptation at the urethral outlet at rest and thus likely to improve the patient's symptoms.

Ceramic particles that have been used in the past as bulking compounds showed to be ineffective owing to the fact that the prior art particles provided insufficient binding areas where tissue could bind. A disadvantage of the known ceramic bulking compounds is therefore that the particles migrate from the area being treated.

Because of the limitations of current materials (resorption and migration), the problem of incontinence is difficult and expensive to control. There is consequently a documented need from the medical community for an injectable composite that would help control incontinence within the Pelvic Floor region/GU system.

### OBJECTS OF THE INVENTION

It is therefore an object of the present invention to provide a tissue bulking agent; a device for bulking a portion of tissue in vivo; and a method and means for treating incontinence with which the aforesaid disadvantages can be overcome or at least minimised. Another object of the present invention is to provide an injectable ceramic composite for use as a tissue bulking agent in the human body.

### SUMMARY OF THE INVENTION

According to a first aspect of the invention there is provided a tissue bulking agent comprising a particulate micro-porous bioinert ceramic material, having interconnected pores.

Further according to the invention the particles are in the form of spheres.

Further according to the invention the micro-pores are exposed on the surface of the spheres, hence rendering the surface of the spheres smooth on a macro-scale, but porous and uneven on a micro-scale to enhance and promote the formation of tissue in the pores and the binding of tissue to the spheres.

Yet further according to the invention, the pores on the surface of each sphere are connected to at least some of the pores inside such sphere via blow-holes and at least some of these internal pores are in turn interconnected, so that in addition to a high porosity, the spheres have a high permeability to gas and liquid.

The particles may have a porosity of between 30% and 70% per volume.

The pores of the particles may have diameters in the range of from 0.3 to 10 micrometer.

The particles may have a diameter larger than 45 micrometer, typically 110 to 600 micrometer.

The particles may be mobilised by mixing the particles with a bio-suitable carrier to render the particles injectable into the body of a patient.

Further according to the invention, the diameter size fractions of the particles do not differ more than 100 micrometer from each other in order to restrict bridging of the particles in a hypodermic needle during injection under pressure into the body of a patient, in use.

The bioinert ceramic material may be selected from the group consisting of sintered aluminium oxide (alumina); sintered zirconium oxide (zirconia); and the combination thereof.

The bioinert ceramic material may be hydroxyapatite.

The particles may be activated prior to injection by impregnation with a substance selected from the group consisting of traces of a bioactive resorbable bioceramic and serum from the blood of a patient.

The particles may be mobilised for injection by mixing the particles with a bio-suitable pharmaceutically acceptable carrier selected from the group consisting of a gel, and a viscous, lubricating liquid.

According to a second aspect of the invention there is provided a device for bulking a portion of tissue in vivo, comprising an effective amount of a tissue bulking agent according to a first aspect of the invention; and applying means for applying the bulking agent to the tissue.

The applying means may be in the form of a hypodermic syringe having an outlet opening of a diameter larger than 110 micrometer.

The bulking agent may be disposed inside the syringe.

According to a third aspect of the invention there is provided use of a tissue bulking agent according to the first aspect of the invention in a method for treating incontinence related disorders in a patient.

According to a fourth aspect of the invention there is provided use of a bulking agent according to the first aspect of the invention in the manufacture of a device for use in a method for treating incontinence related disorders in a patient.

According to a fifth aspect of the invention there is provided a method of treating incontinence related disorders in a patient including the step of locating an effective amount of a tissue bulking agent according to the first aspect of the invention in an affected area of the patient in need thereof.

The step of locating the bulking agent in the affected area may include the further step of injecting the bulking agent into the urinary sphincter, to increase resistance to the flow of urine.

The method may include the further step of moistening the bulking agent with blood drawn from the patient into a syringe containing the agent, such that the red blood cells accumulate on the surface of the particles and the serum is drawn into and stored inside the particles.

The method may include the further step of activating the particles prior to injection by impregnation with a substance selected from the group consisting of traces of a bioactive resorbable biocerarnic and serum from the blood of a patient.

The method may include the further step of mobilising the particles for injection by mixing the particles with a bio-suitable pharmaceutically acceptable carrier selected from the group consisting of a gel, and a viscous, lubricating liquid.

According to a sixth aspect of the invention there is provided a method of bulking a portion of tissue of a human body including the step of introducing to that portion a volume of the bulking agent according to a first aspect of the invention.

The method may include the further step of injecting the bulking agent subcutaneously.

Alternatively, the method may include the further step of injecting the bulking agent into the tissue itself.

The invention will now be described further by way of a non-limiting example of a preferred embodiment of the invention.

The bulking agent according to the present invention is prepared by a method including the steps of:
milling solid alumina (Al₂O₃) into powder (1 micrometer diameter particles);
adding a combustible substance known in the trade of manufacturing porous ceramic structures to the powder;
mixing the powder with water to form a paste or slurry;
forming solid spherical particles from the slurry by applying methods known in the art;
sintering the spherical particles at a temperature of 1350° C. to form inert micro-porous ceramic spheres having pore sizes of between 0.3 to 10 micrometers and diameter of between 110 and 600 micrometers; and
screening the spheres into pre-selected size ranges, with the respective diameters of spheres in a particular range not differing more than 100 micrometer from each other.

The applicants have found that a bulking agent according to the present invention meets most of the clinical requirements for the successful treatment of urinary incontinence in that the bulking agent, inter alia:
is an immobile, permanent bulking agent;
conforms to physical requirements for placement by injection;
is non-immunogenic, hypoallergenic and non-antigenic;
bio-compatible, non-resorbable, with no measurable inflammatory response;
provides satisfactory and rapid wound healing in the applied area with adequate or minimal fibrotic ingrowths;
retains tissue bulking/enhancement and in some cases replacement over prolonged periods of time;
when injected into the urethral sphincter of patients suffering from pressure incontinence of urine, the appropriate volume of ceramic spheres increases the intra-urethral pressure so as to render the treated patients more continent of urine;
includes spheres having particle sizes above 100 microns and surface roughness or pores exposed on the surface that instigate adhesion to host tissue; and
provides synthesis in functionality between a carrier and the injectable composite material.

In use, the bulking agent is disposed inside a syringe having an outlet and needle attached to the outlet with a bore having a diameter larger than 0.6 mm. The bulking agent is then activated by impregnating the spheres with traces of a bloactive resorbable bioceramic such as tricalcium phosphate. The spheres are mobilised for injection by mixing the spheres with a bio-suitable carrier gel or a viscous, lubricating carrier liquid.

Thereafter, the activated and mobilised bulking agent is injected into the tissue in the area to be treated or bulked, such as the pelvic floor or urinary sphincter.

The spheres could alternatively be activated and mobilised by the in situ impregnation with serum from the blood of a patient. This includes the moistening of the bulking agent with blood drawn from the patient into the syringe containing the agent, such that the red blood cells accumulate on the surface of the spheres and the serum is drawn into and stored inside the spheres. The applicants foresee that this will stimulate tissue forming on the surface even more and will reduce any imminogenic reactions to the bulking agent even further.

The applicants further foresee that the bulking agent according to the invention could be manufactured and prepared for application at a relatively low cost.

It will be appreciated that the application of the bulking agent according to the present invention is not limited to use in the treatment of incontinence related disorders, but could be applied anywhere in the human body where a bulking agent is required, such as in reconstructive surgery following damage to tissue; and in cosmetic surgery where the volume of certain body parts such as lips are to be increased.

The characteristics of the bulking agent of the present invention, such as, inter alia, the low cost of manufacture; the safety; stability; immobility after application; and the ease of application thereof, not only overcomes the disadvantages of the prior art, but also renders the bulking agent and the method of the invention suitable and available to a wide range of patients including those suffering from incontinence related disorders.

The description of the embodiments of the present invention is given above for the understanding of the present invention. It will be appreciated further that the invention is not limited to the particular embodiments described herein, but is capable of various modifications and rearrangements as will be apparent to those skilled in the art without departing from the scope of the appended claims.

1. A tissue bulking agent comprising a particulate micro-porous bioinert ceramic material, having interconnected pores.

2. A tissue bulking agent according to claim 1 wherein the particles are in the form of spheres.

3. A tissue bulking agent according to claim 2 wherein the micro-pores are exposed on the surface of the spheres, hence rendering the surface of the spheres smooth on a macro-scale, but porous and uneven on a micro-scale to enhance and promote the formation of tissue in the pores and the binding of tissue to the spheres.

4. A tissue bulking agent according to claim 3 wherein the pores on the surface of each sphere are connected to at least some of the pores inside such sphere via blow-holes and at least some of these internal pores are in turn interconnected, so that in addition to a high porosity, the spheres have a high permeability to gas and liquid.

5. A tissue bulking agent according to claim 1 wherein the particles have a porosity of between 30% and 70% per volume.

6. A tissue bulking agent according to claim 1 wherein the pores of the particles have diameters in the range of from 0.3 to 10 micrometer.

7. A tissue bulking agent according to claim 1 wherein the particles have a diameter larger than 45 micrometer.

8. A tissue bulking agent according to claim 7 wherein the particles have a diameter of 110 to 600 micrometer.

9. A tissue bulking agent according to claim 1 wherein the particles are mobilised by mixing the particles with a bio-suitable carrier to render the particles injectable into the body of a patient.

10. A tissue bulking agent according to claim 9 wherein the particles are mobilised for injection by mixing the particles with a bio-suitable pharmaceutically acceptable carrier selected from the group consisting of a gel, and a viscous, lubricating liquid.

11. A tissue bulking agent according to claim 9 wherein the diameter size fractions of the particles do not differ more than 100 micrometer from each other in order to restrict bridging of the particles in a in a hypodermic needle during injection under pressure into the body of a patient, in use.

12. A tissue bulking agent according to claim 9 wherein the particles are activated prior to injection by impregnation with a substance selected from the group consisting of traces of a bioactive resorbable bioceramic and serum from the blood of a patient.

13. A tissue bulking agent according to claim 1 wherein the bioinert ceramic material is selected from the group consisting of sintered aluminium oxide (alumina); sintered zirconium oxide (zirconia); and the combination thereof.

14. A tissue bulking agent according to claim 1 wherein the bioinert ceramic material is hydroxyapatite.

15. A device for bulking a portion of tissue in vivo, comprising an effective amount of a tissue bulking agent according to claim 1; and applying means for applying the bulking agent to the tissue.

16. A device according to claim 15 wherein the applying means is in the form of a hypodermic syringe having an outlet opening of a diameter larger than 110 micrometer.

17. A device according to claim 16 wherein the bulking agent is disposed inside the syringe.

18. Use of a tissue bulking agent according to claim 1 in a method for treating incontinence related disorders in a patient.

19. Use of a tissue bulking agent according to claim 1 in the manufacture of a device for use in a method for treating incontinence related disorders in a patient.

20. A method of treating incontinence related disorders in a patient including the step of locating an effective amount of a tissue bulking agent according to claim 1 in an affected area of the patient in need thereof.

21. A method according to claim 20 wherein the step of locating the bulking agent in the affected area includes the further step of injecting the bulking agent into the urinary sphincter, to increase resistance to the flow of urine.

22. A method according to claim 20 which includes the further step of moistening the bulking agent with blood drawn from the patient into a syringe containing the agent, such that the red blood cells accumulate on the surface of the particles and the serum is drawn into and stored inside the particles.

23. A method according to claim 22 which includes the further step of activating the particles prior to injection by impregnation with a substance selected from the group consisting of traces of a bioactive resorbable bioceramic and serum from the blood of a patient.

24. A method according to claim 23 which includes the further step of mobilising the particles for injection by mixing the particles with a bio-suitable pharmaceutically acceptable carrier selected from the group consisting of a gel, and a viscous, lubricating liquid.

25. A method of bulking a portion of tissue of a human body including the step of introducing to that portion a volume of the bulking agent according to claim 1.

26. A method according to claim 25 which includes the further step of injecting the bulking agent subcutaneously.

27. A method according to claim 25 which includes the further step of injecting the bulking agent into the tissue itself.

28-32. (canceled)
